(19) United States
(12) Patent Application Publication
(10) Pub. No.: US 2013/0172800 A1
(43) Pub. Date: Jul. 4, 2013

(54) Brace Affixing Lower Leg Cuff

(71) Applicant: Mike Ritzmann, Galveston, TX (US)

(72) Inventor: Mike Ritzmann, Galveston, TX (US)

(73) Assignee: Mike Ritzmann, Galveston, TX (US)

(21) Appl. No.: 13/601,375

(22) Filed: Aug. 31, 2012

Related U.S. Application Data
(60) Provisional application No. 61/531,094, filed on Sep. 6, 2011.

(51) Int. Cl. A61F 5/01

(52) U.S. Cl. CPC A61F 5/0123
USPC 602/26

(57) ABSTRACT

In one embodiment of the invention, a lower leg cuff integrates with existing leg braces and prevents said leg braces from slipping down wearer's leg. The cuff is circumferentially placed around the lower leg in an above-the ankle or supramalleolar position with the lowest aspect of the cuff able to accommodate, in preformed apertures, the topmost portions of both the tibial and lateral malleoli. The leg cuff, thus “locked” into position on the leg has tabs which connect via hook and loop fastener with the lowest member of an in-situ leg brace. This arrangement will support the brace's longitudinal placement on the leg and due to the cuff's inability to slide onto or over the two bony protuberances of the lower leg the cuff is unable to migrate and thus will prevent the longstanding problem of slippage of the wearer's orthosis along the vertical axis of the leg.

### CROSS REFERENCE TO RELATED APPLICATION

This application claims the benefit of priority from U.S. provisional application 61/531,094 filed on Sep. 6, 2011.

### REFERENCES

U.S. PATENT DOCUMENTS

| | | |
|---|---|---|
| 5,372,572 | December 1994 | Tamagni |
| 7,507,215 | March 2009 | Ryan |
| 5,074,290 | December 1991 | Harris |
| 6,635,024 | October 2003 | Hatton, et. al. |
| 7,410,471 | August 2008 | Campbell |
| 3,900,898 | August 1975 | Ackerman |
| 6,764,457 | July 2004 | Hogg |
| 7,896,827 | March 2011 | Ingimundarson |
| 8,016,781 | September 2011 | Ingimundarson |
| 5,242,378 | September 1993 | Baker |
| 4,287,884 | September 1981 | Applegate |
| 7,128,723 | October 2006 | Doty |
| 5,599,283 | February 1997 | Lindenmeyer |
| 6,524,265 | February 2003 | Hogg |
| US2010/0010409 | February 2009 | Bejarano |

### BACKGROUND

The invention is a padded cuff that rests against the tops of the tibial and fibular malleoli in the supramalleolar position, away from the ankle joint, surface of the foot and all articulating surfaces and major muscle groups. This placement on the lower leg, above the bony protuberances of the distal tibia and fibula, will do a very effective job of keeping the cuff in place and not disrupting the underlying anatomy. The cuff has a liner designed to accept the superior-most aspects of both the medial malleolus and lateral malleolus, and will yield far greater and far more permanent fixation and stability than a traditional system of bindings. This permits the wearer to maintain the attached brace's proper positioning relative to the knee and avoid having to excessively tighten the brace's leg bindings in an attempt to prevent vertical migration of the brace.

Orthoses suffer from a common deficiency—they move relative to the limb to which they are attached. In regards to a leg brace, manufacturers use various strapping systems in an attempt to keep the device in place. Typically this involves a series of buckled hook and loop fasteners. This approach has many deficiencies. The musculature that the straps are overlying, (quadriceps muscle groups and the gastrocnemius muscle of the calf) are changing shape and size with every step the brace wearer takes. The failure of such binding systems to yield a permanent fixture is inherent in their design. The mere shape of the quadriceps muscles, the superior aspect being wider and more massive, ensures that as it contracts and expands it will drive an overlying binding strap downward toward the knee. As the orthosis slips downward, the hinge joint on the device becomes misaligned with the wearer's knee joint. This causes discomfort and dysfunction at best. In an attempt to stimy the migration, the wearer will make the bindings ever tighter which can lead to muscular dysfunctions like cramping and spasticity. An even more significant medical concern—the threat of a deep vein thrombosis—is well acknowledged in cases of ligatures being applied to the legs. DVTs are often seen as an after effect of surgeries where tourniquet cordage is applied to the limbs. The pulmonary emboli that can result from deep vein thromboses are well-documented killers. Some brace manufacturers are attempting to address the issue by putting a pad between the knee joint and the knee itself and claiming that the condyle support being afforded will keep their braces in situ.

### BRIEF SUMMARY OF THE INVENTION

The invention is held in place with a buckle and padded hook and loop backed strapping that is integral to the collar. By virtue of having the cuff fit above, or superiorly, to the medial malleolus and lateral malleolus, it is prevented from “sliding down” the leg. Tabs on the lateral aspects of the cuff extend high enough to allow hook and loop fasteners on the tab and lower portion of the wearer's brace to substantially overlap and join. This locks the wearer's brace to the cuff and fixes the brace's vertical position on the leg and will not allow up and down movement, or migration, relative to the leg. The brace is not able to migrate vertically as it is exposed to movement, sweat and contact.

### BRIEF SUMMARY OF THE DRAWINGS

Drawing Page 1 presents a lateral view of the leg with lower leg cuff in place.

Drawing Page 2 is a frontal view of the lower leg cuff. Visible in profile are the tab and the lower support member of a brace, also in profile view. The tab and support member are joined by hook and loop fastener.

Drawing Page 3 is a view from the bottom of the lower leg cuff. The widened apertures that sit above the malleoli are visible as well as the hook and loop covered fastener and buckle.

### DETAILED DESCRIPTION

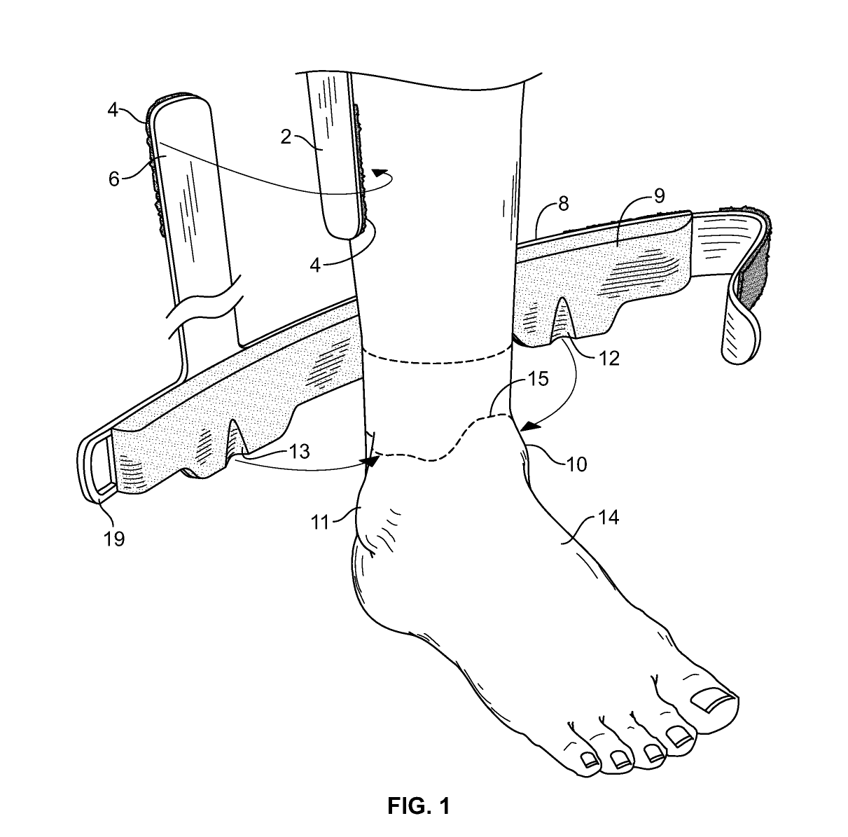

FIG. 1 depicts one embodiment of a cuff that attaches to the wearer's lower leg for the purpose of attaching to, and preventing the movement of, the wearer's leg brace. The lower leg cuff will be wide enough to establish and maintain a comfortable but substantial fixture around the distal portions of the tibia and fibula. The cuff, made of nylon or otherwise suitable materials, will incorporate a liner 9 that forms two rigid concavities, one on the medial side of the leg 12 and one on the lateral side of the leg 13 that will accept the superior-most aspect of the medial malleolus 10 and the lateral malleolus 11. The liner 9 will be padded with neoprene, moleskin and like materials.

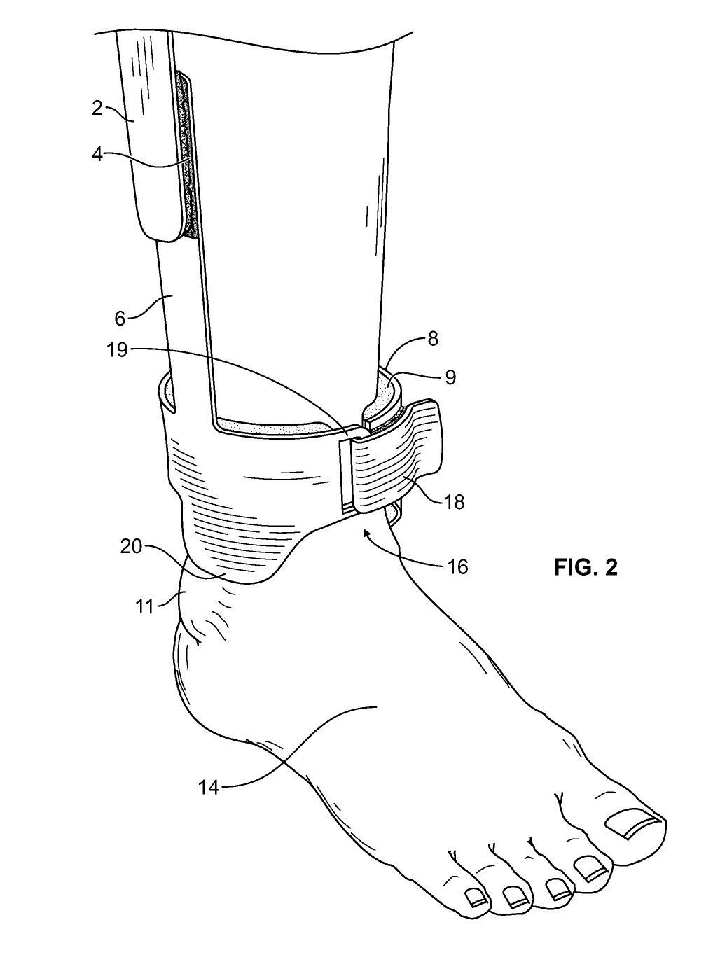

Referring to FIG. 2, the tension of the cuff around the wearer's lower leg can be adjusted by means of a hook and loop backed extension 18 of the cuff's liner 8. This extension can be fed through the integral buckle 19 and secured to itself with the hook and loop fastener. Arising from the cuff and projecting superiorly is a tab, constructed of any material that is lightweight, appropriately flexible and of sufficient strength to support the rigors of the application. Carbon and fiberglass composites as well as various plastics would be suitable choices. The tab, several inches in length, is backed with hook and loop fastener and mates 4 with a substantially similar tab 2 that is affixed to wearer's leg brace and which extends inferiorly along the leg to meet and attach 4 to cuff tab 6. This mating system allows the wearer to find optimal placement for their leg orthosis and then, using the infinite adjustability of the hook and loop-backed tabs, secure the tabs together 4 and lock the orthosis into position, thereby eliminating vertical creep or migration of the wearer's leg brace and maintaining optimal placement of the brace's hinge relative to the wearer's knee.

Referring again to FIG. 1, the lower margin of the cuff 15 is superimposed on the wearer's foot 14 to demonstrate the abundance of clearance between the foot's upper instep and the lowest margin of the cuff 15. Turning to FIG. 2 we can better see the space 16 that is occupied by the upper instep. This area 16 is free of obstruction and interference from the cuff, thereby enabling the wearer to move the foot entirely independently of the cuff which is situated above the fixed ankle bones (10 and 11 in FIG. 1).

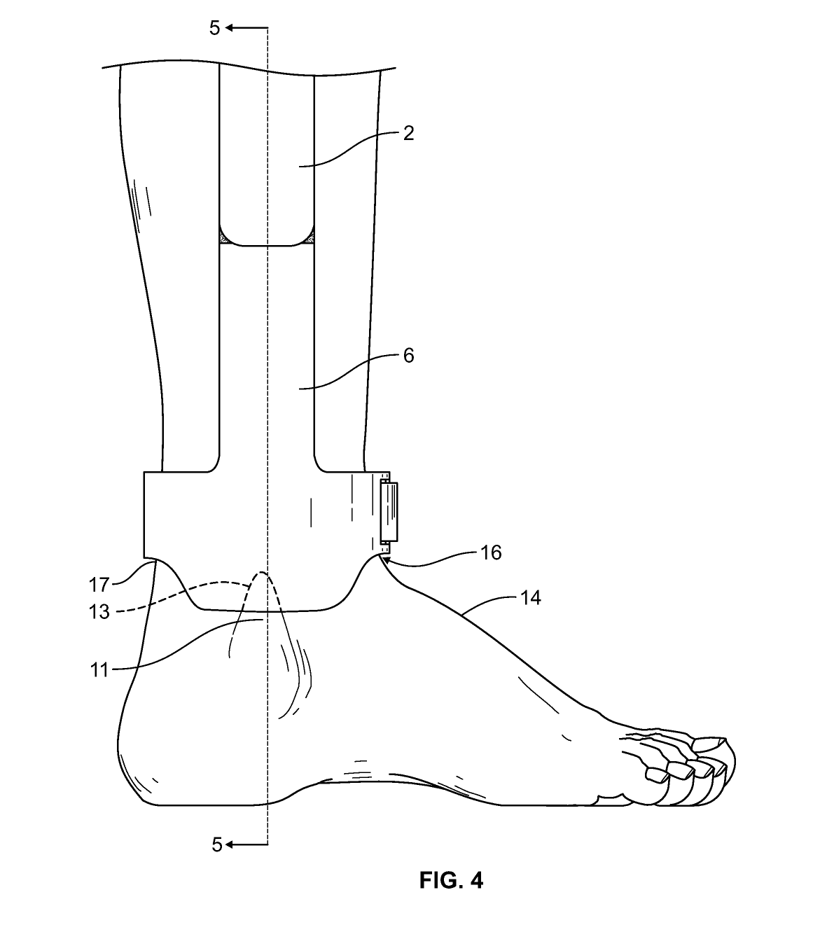

FIG. 4 also illustrates this most important concept wherein one can see that the space of the upper instep 16 is free of interference from the cuff. Additionally, the area of the Achilles tendon, superior to the heel 17, is also free of obstruction and interference from the brace. Also in FIG. 4, the hashed area 13 represents the aperture formed into the cuff's liner that accepts the superior-most aspect of the ankle bone, the lateral malleolus in this depiction. By keeping the apertures 12 and 13 of FIG. 1 small enough to accept no more than the superior-most aspect of the malleoli, the cuff is unable to be forced downward toward the foot as, moving inferiorly, the size of the ankle bones expands considerably and will prevent any vertical migration of the cuff. This design limits the cuff's ability to “slide” down the leg and also removes the possibility of rotation around the long axis of the leg.

Essentially, what is provided is a stable, fixed platform that will not migrate from its position and will allow an almost infinite variety of attachments to hold the wearer's leg brace in situ without suffering from movement dependent on an articulable relationship with the foot. The foot and the cuff/brace arrangement will have no interplay and as such will function as two discrete systems with no movement being transmitted from the foot to the cuff/brace arrangement during ambulation or other articulations of the foot.

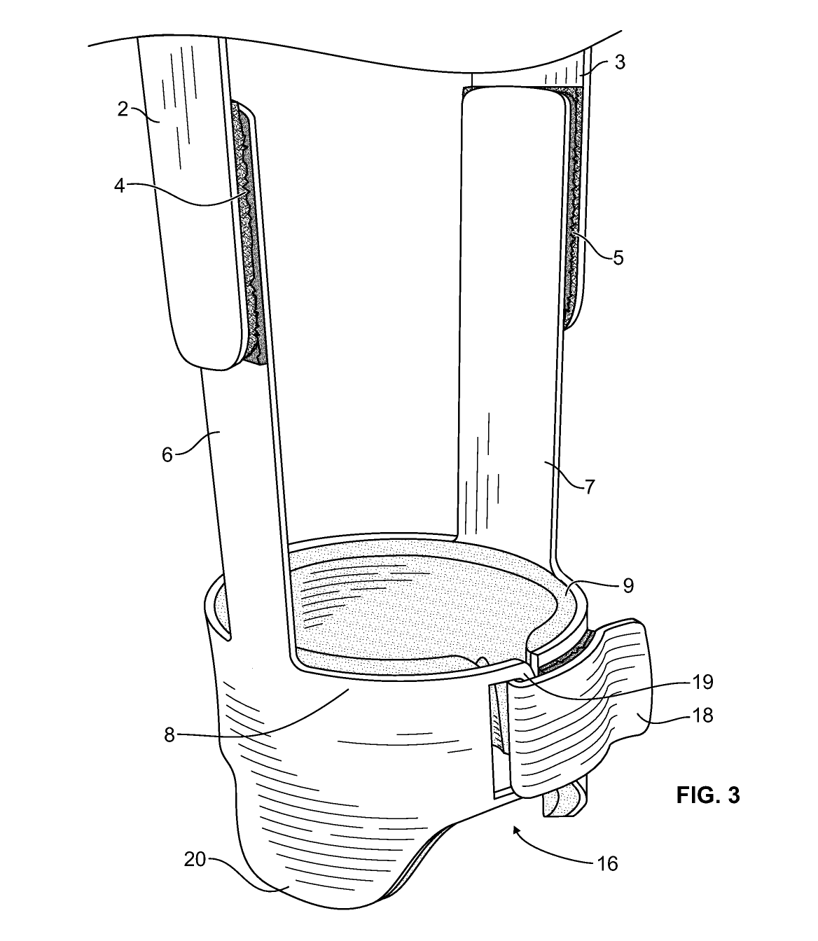

FIG. 3 shows another embodiment of the design in which a second, and medial, tab 7 extends superiorly from the cuff and, fixed with hook and loop fastener, allows a similarly infinite number of attachment points 5 to an inferiorly extending tab 3 which is affixed to the wearer's brace. In this embodiment of the design all parameters of the cuff, excepting the additional tabs, are similar to, if not identical to the herein described embodiment of the cuff.

Also depicted in FIG. 3 is a representation of the space occupied by the upper instep, 16, wherein we can see that the foot will freely undergo dorsiflexion with no interference from the cuff. For contrast, 20 is the portion of the cuff that extends inferiorly to an extent great enough to hold the liner aperture (in FIG. 4, 13) firmly against the superior-most aspect of the malleoli. This placement ensures that the foot can articulate in every direction, not just dorsiflex and plantar flex, with no consideration of affecting the cuff and thereby the attached brace.

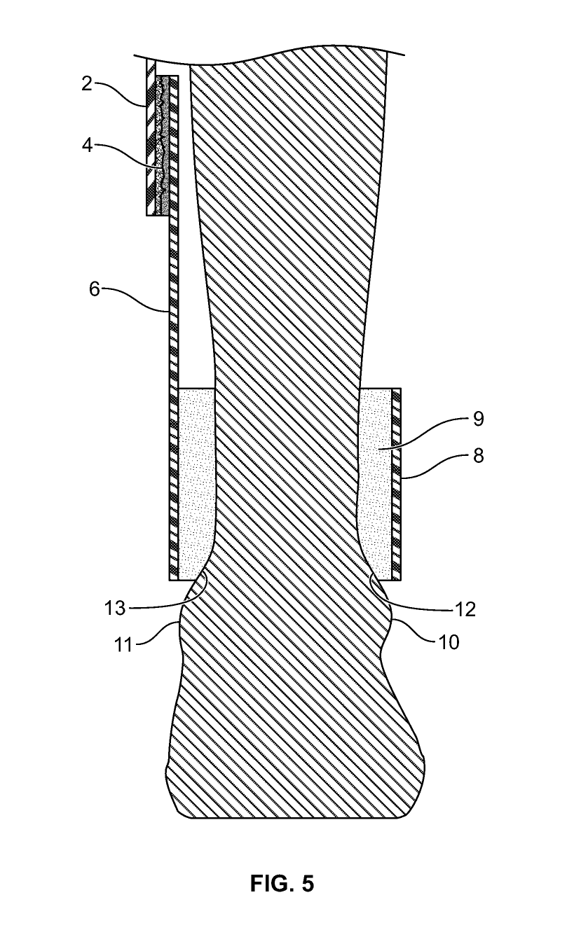

FIG. 5 shows a frontal view of the wearer's leg with a directional reference included in FIG. 4. The medial malleolus is shown supporting the preformed liner 8 as the liner 9 is compressed circumferentially onto the leg by the encircling collar 8. The lateral malleolus can also be seen with the preformed liner aperture 13 accepting the superior-most aspect of the lateral malleolus. Referring to the construction of the cuff's collar 8, it will be formed of a pliable material such as, but not necessarily, nylon and will have integral to it a semi-rigid liner 9 that can be glued, sewn or otherwise permanently affixed to the interior of the collar. This liner will have the apertures 12 and 13 of FIG. 1, that will sit just superior to the wearer's malleoli. The tab 6, or tabs 6 and 7, can be extruded from the collar 8 or the liner material 9 or in other embodiments of this design the tabs, 6 and 7, could be glued, sewn or otherwise affixed to the collar 8 or liner 9 so as to promote cost effectiveness, utility and durability.

Referring again to FIG. 4 to illustrate the concept of freedom of movement between the foot and the cuff, we can see that the cuff is applied superiorly to the fixed distal ends of the tibia and fibula. Additionally, the void 16 allows full dorsiflexion with no contact between the foot 14 and the cuff. Additionally, the area of the Achilles tendon, superior to the heel 17, is also free of obstruction and interference from the brace. Also in FIG. 4, the hashed area 13 represents the aperture formed into the cuff's liner 9 that accepts the superior-most aspect of the ankle bone, the lateral malleolus in this depiction. By keeping the apertures 12 and 13 of FIG. 1 small enough to accept no more than the superior-most aspect of the malleoli, the cuff is unable to be forced downward toward the foot as, moving inferiorly, the size of the ankle bones expands considerably and will prevent any vertical migration of the cuff. This design limits the cuff's ability to “slide” down the leg and also removes the possibility of rotation around the long axis of the leg.

Essentially, what is provided is a stable, fixed platform that will not migrate from its position and will allow an almost infinite variety of attachments to hold the wearer's leg brace in situ without suffering from movement dependent on an articulable relationship with the foot. The foot and the cuff-brace arrangement will have no interplay and as such will function as two discrete systems with no movement being transmitted from the foot to the cuff-brace arrangement during ambulation or other articulations of the foot.

I claim:

1. A lower leg cuff for attaching to, and preventing vertical movement of, orthopedic leg braces, comprising: an encompassing collar, a liner encompassing two rigid cavities to sit above the tibial and fibular malleoli, a fastening system, at least one semi rigid tab extending superiorly toward the knee.

2. The cuff of claim 1 wherein semi rigid cavities are formed of a plastic or otherwise suitable material and are created and formed as an integral part of the semi rigid plastic, or otherwise suitable material, liner that is bonded to the interior facing of the collar.

3. The cuff of claim 1 wherein the two semi rigid cavities are shaped so as to fit, in the context of the collar, around the wearer's leg and specifically, supramalleolarly to the tibial and fibular malleoli with the cavities' widest aperture being at the bottom or inferior most edge of the collar thereby permitting the cavities to accept a small portion of the superior-most aspects of both the tibial and fibular malleoli.

4. The cuff of claim 1 wherein the two semi rigid cavities are sized and shaped so as to prevent the acceptance of more than the superior-most aspect of the tibial and fibular malleoli into the cavities thereby keeping the cuff well above the foot and any articulating areas thereby preventing interference between the brace and/or cuff and any articulating aspects of the ankle or foot.

5. The cuff of claim 1 wherein cuff is in contact with only the superior-most aspects of the tibial and fibular malleoli, cuffs placement will in no way impact, nor be impacted by, the articulation of the foot and ankle during ambulation which will ensure that attached orthopedic brace remains in a fixed position and does not vertically migrate relative to the leg.

6. A means of attaching lower leg cuff to leg brace/orthosis by means of one or two protuberances, or tabs, of plastic or otherwise suitable material, that are permanently attached to cuff and extend superiorly, or toward the knee, on the lateral side of the leg in the case of one tab, or on the lateral and medial sides of the leg in the case of two tabs.

7. The tab(s) of claim 6 wherein the tab, or tabs, are dimensionally sized to allow secure anchoring of tab(s) in the collar, allow ample height to unite tab(s) with the lower brace member and of such sizing as to establish ample rigidity in the tab(s) to withstand without deformation any loading by the attached brace.

8. The tab(s) of claim 6 wherein tab(s) will be surfaced with hook and loop fastener that will bond with hook and loop fastener applied to the lower member of the wearer's brace.

9. A lower leg cuff wherein said cuff supports any attached orthopedic brace; said brace can be installed and maintained in position with substantially looser circumferential bindings than would otherwise be possible, thereby reducing the risk of ligature-induced deep vein thrombosis and subsequent pulmonary emboli.

10. The cuff of claim 9 wherein said cuff's construction of semi rigid plastic or otherwise suitable materials including but not limited to nylon, cloths, fiber reinforced plastics, buckles and hook and loop fastener, ensures cuffs resistance to deformation and brittle failure under conditions of rigorous physical application such as athletic competitions.

11. The cuff of claim 9 wherein said cuff's construction of materials including, but not limited to, nylon, cloths, fiber reinforced plastics, buckles and hook and loop fastener, ensures a margin of safety during athletic competitions or occupational undertakings whereby said cuff does not present metallic fasteners, struts, clamps or protuberances that may present an injury hazard to wearer or other participants in said athletic events or occupational undertakings.
